(19) United States
(12) Patent Application Publication
(10) Pub. No.: US 2023/0285657 A1
(43) Pub. Date: Sep. 14, 2023

(54) PERFUSION TARGET INTERNAL PRESSURE ESTIMATION METHOD, PERFUSION SYSTEM, AND LIVING BODY INTERNAL PRESSURE ESTIMATION METHOD

(71) Applicant: OLYMPUS MEDICAL SYSTEMS CORP., Tokyo (JP)

(72) Inventor: Hiroki KAZUNO, Koganei-shi (JP)

(73) Assignee: OLYMPUS MEDICAL SYSTEMS CORP., Tokyo (JP)

(21) Appl. No.: 18/119,468

(22) Filed: Mar. 9, 2023

Related U.S. Application Data
(60) Provisional application No. 63/319,084, filed on Mar. 11, 2022.

(51) Int. Cl. A61M 1/00

(52) U.S. Cl. CPC A61M 1/72; A61M 1/77; A61M 2205/3334; A61M 2205/3344; A61M 2205/3393

(57) ABSTRACT

A perfusion target internal pressure estimation method includes, at a time of a first operation for feeding liquid and not suctioning the liquid, acquiring a liquid feed flow rate in a liquid feed passage and pressure in a suction passage, at a time of a second operation for feeding the liquid and suctioning the liquid, acquiring a liquid feed flow rate in the liquid feed passage and a suction flow rate in the suction passage, subtracting the suction flow rate from the liquid feed flow rate to acquire a flow rate difference, and acquiring, based on a regression formula of the liquid feed flow rate and the pressure in the suction passage at the time of the first operation, an estimation value of an internal pressure of a perfusion target at the time of the second operation from the flow rate difference.

### CROSS-REFERENCE TO RELATED APPLICATION

This application is based on and claims priority under 35 U.S.C. § 119 to U.S. Provisional Application No. 63/319,084, filed Mar. 11, 2022, the entire contents of which are incorporated herein by reference.

### BACKGROUND

### 1. Technical Field

The present disclosure relates to a perfusion target internal pressure estimation method, a perfusion system, and a living body internal pressure estimation method for performing liquid feed and suction to generate perfusion.

### 2. Description of the Related Art

For example, there has been proposed a perfusion system that, for a perfusion target including a dead end structure like an inside of a kidney, fractures a calculus in the perfusion target into fractured stone pieces using a laser, performs liquid feed and suction in combination to generate perfusion in the perfusion target, and collects the calculus from the perfusion target. Such a perfusion system can be put to practical use using, for example, an endoscope including an optical fiber channel, a liquid feed channel, and a suction channel.

Incidentally, since a pressure inside an organ (an internal pressure) to be a perfusion target changes according to a balance of the liquid feed and the suction, the perfusion system desirably can grasp the internal pressure when implementing perfusion.

For example, in FIG. 2, FIG. 9, and the like of Japanese Patent Application Laid-Open Publication No. 2020-518342, a technique for, in a fluid management system including a perfusion mechanism, providing a pressure sensor at a distal end of a flexible elongated shaft extending from a scope and directly detecting an internal pressure in, for example, a kidney in a body is described.

### SUMMARY

A perfusion target internal pressure estimation method according to an aspect of the present disclosure includes: at a time of a first operation for feeding liquid to a perfusion target with a liquid feed passage and not suctioning the liquid from the perfusion target with a suction passage, acquiring a liquid feed flow rate in the liquid feed passage and pressure in the suction passage; at a time of a second operation for feeding the liquid to the perfusion target with the liquid feed passage and suctioning the liquid from the perfusion target with the suction passage, acquiring a liquid feed flow rate in the liquid feed passage and a suction flow rate in the suction passage; subtracting the suction flow rate from the liquid feed flow rate to acquire a flow rate difference; and acquiring, based on a regression formula of the liquid feed flow rate and the pressure in the suction passage at the time of the first operation, an estimation value of an internal pressure of the perfusion target at the time of the second operation from the flow rate difference.

A perfusion system according to an aspect of the present disclosure includes a first pump configured to feed liquid to a liquid feed passage; a first flowmeter configured to detect a liquid feed flow rate in the liquid feed passage; a second pump configured to suction the liquid from a suction passage; a second flowmeter configured to detect a suction flow rate in the suction passage; a pressure gauge configured to detect pressure in the suction passage; and a processor configured to control the first pump and the second pump based on information from the first flowmeter, the second flowmeter, and the pressure gauge. At a time of a first operation of controlling the first pump to feed the liquid to the liquid feed passage and a perfusion target and of controlling the second pump not to suction the liquid from the suction passage and the perfusion target, the processor is configured to perform: acquiring the liquid feed flow rate detected by the first flowmeter and the pressure in the suction passage detected by the pressure gage, and at a time of a second operation of controlling the first pump to feed the liquid to the liquid feed passage and the perfusion target and of controlling the second pump to suction the liquid from the suction passage and the perfusion target, the processor is configured to perform: acquiring the liquid feed flow rate detected by the first flowmeter and the suction flow rate detected by the second flowmeter; subtracting the suction flow rate from the liquid feed flow rate to acquire a flow rate difference; and acquiring, based on a regression formula of the liquid feed flow rate and the pressure in the suction passage at the time of the first operation, an estimation value of an internal pressure of the perfusion target at the time of the second operation from the flow rate difference.

A perfusion system according to an aspect of the present disclosure includes: a liquid feed passage; a pump configured to feed liquid to the liquid feed passage; a pressure gauge provided on the liquid feed passage; a first flowmeter provided on the liquid feed passage; and a processor including hardware, the processor: when the liquid is fed to the liquid feed passage by the pump, subtracting, from pressure in the liquid feed passage detected by the pressure gauge, a pressure drop corresponding to a liquid feed flow rate in the liquid feed passage detected by the first flowmeter and acquiring an estimation value of pressure at a distal end of the liquid feed passage.

A living body internal pressure estimation method according to an aspect of the present disclosure includes: disposing a distal end of a liquid feed passage and a distal end of a suction passage in a living body; performing a first operation for feeding liquid to the living body with the liquid feed passage and not suctioning the liquid from the living body with the suction passage; performing a second operation for feeding the liquid to the living body with the liquid feed passage and suctioning the liquid from the living body with the suction passage; and acquiring, based on a regression formula of a liquid feed flow rate of the liquid feed passage and pressure in the suction passage at a time of the first operation, an estimation value of an internal pressure of the living body at a time of the second operation from a flow rate difference obtained by subtracting a suction flow rate of the suction passage from a liquid feed flow rate of the liquid feed passage at the time of the second operation.

### BRIEF DESCRIPTION OF THE DRAWINGS

FIG. 1 is a diagram showing a configuration example of an endoscope system to which a perfusion system in a first embodiment of the present disclosure is applied;

FIG. 2 is a diagram showing a configuration example of the perfusion system in the first embodiment;

FIG. 3 is a perspective view showing a state of a distal end portion of an insertion section of an endoscope through which a laser probe is inserted in the first embodiment;

FIG. 4 is a diagram showing a state of the distal end portion of the insertion section of the endoscope after the laser probe is pulled out;

FIG. 5 is a timing chart showing a flow of processing for performing a first operation and calculating a regression formula prior to performing a second operation in the perfusion system in the first embodiment;

FIG. 6 is a timing chart showing a flow of processing of the perfusion system at a time when the first operation is performed in order to calculate the regression formula in the first embodiment;

FIG. 7 is a graph showing an example of a regression formula calculated by a processor;

FIG. 8 is a flowchart showing processing for switching display of an internal pressure in the perfusion system in the first embodiment; and

FIG. 9 is a time chart showing an example in which pressure detected by a pressure gauge changes when a liquid feed flow rate and a suction flow rate do not change in the first embodiment.

### DETAILED DESCRIPTION

In general, a urinary tract such as a kidney, a ureter, a bladder, and a urethra has a small diameter, it is sometimes difficult in itself to dispose a pressure gauge such as a pressure sensor in an organ to be a perfusion target or, even if the pressure gauge can be disposed, the pressure gauge sometimes hinders calculus collection by perfusion. When a calculus in a liquid flow frequently collides with the pressure gauge, detected pressure is sometimes inaccurate. Further, since a small and small-diameter pressure sensor is high in a unit price, the pressure sensor is a cause of an increase in manufacturing cost of an entire perfusion system.

According to an embodiment explained below, it is possible to provide a perfusion target internal pressure estimation method, a perfusion system, and a living body internal pressure estimation method that can grasp an internal pressure of a perfusion target without disposing a pressure gauge in the perfusion target.

An embodiment of the present disclosure is explained below with reference to the drawings. However, the present disclosure is not limited by the embodiment explained below.

Note that, in description of the drawings, same or corresponding elements are denoted by the same reference numerals and signs as appropriate. It needs to be noted that the drawings are schematic and relations among lengths of respective elements, ratios of the lengths of the respective elements, quantities of the respective elements, and the like in one drawing are sometimes different from realities in order to simplify explanation. Further, among a plurality of drawings, portions having different relations among lengths of the portions and ratios of the lengths are sometimes included.

### First Embodiment

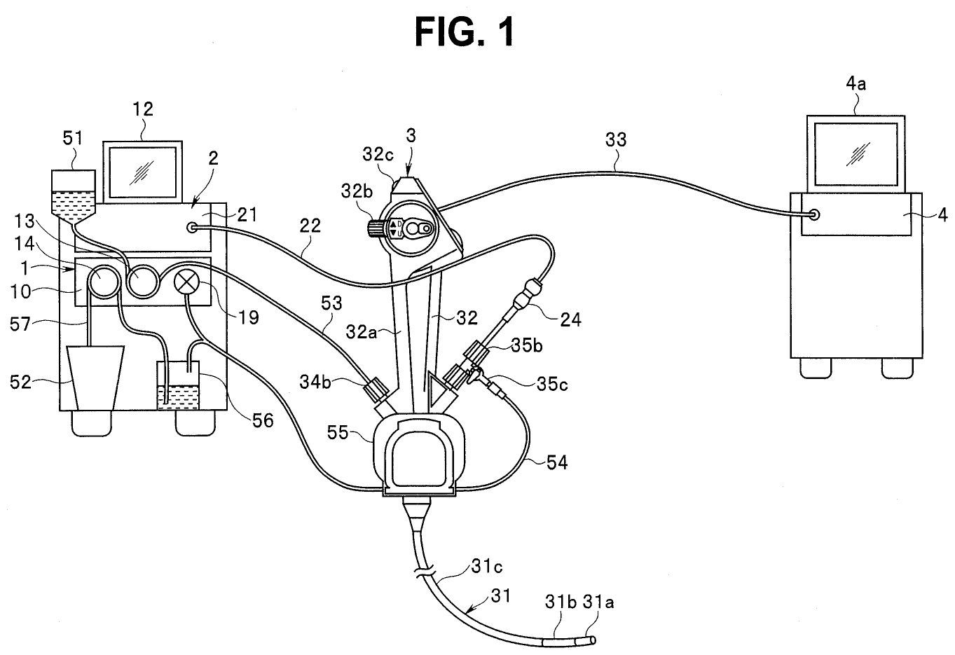

FIG. 1 to FIG. 9 show a first embodiment of the present disclosure. FIG. 1 is a diagram showing a configuration example of an endoscope system to which a perfusion system 1 in the first embodiment of the present disclosure is applied.

As shown in FIG. 1, the endoscope system in the present embodiment includes the perfusion system 1, a laser system 2, an endoscope 3, and an endoscope control apparatus 4.

The endoscope 3 is a device that performs observation and treatment of a subject. The endoscope 3 includes an elongated insertion section 31 to be inserted into the subject, an operation section 32 provided on a proximal end side of the insertion section 31, and a universal cable 33 extended from the operation section 32. Note that the subject into which the insertion section 31 is inserted is assumed to be a living body such as a person or an animal. Specific examples of a perfusion target 90 by the perfusion system 1 is a kidney (a renal pelvis, a renal calix, and the like), a ureter, a bladder, a urethra, and the like.

The insertion section 31 includes a distal end portion 31a, a bending section 31b, and a tubular section 31c in order from a distal end toward a proximal end.

The distal end portion 31a includes an observation system and an illumination system. The observation system forms an optical image of the subject with an objective optical system and photoelectrically converts the optical image with an image pickup device to generate an image pickup signal. The illumination system transmits illumination light with, for example, a light guide and irradiates the subject with the illumination light from a distal end of the light guide. A signal line and the light guide connected to the image pickup device are disposed in the insertion section 31, the operation section 32, and the universal cable 33 and connected to the endoscope control apparatus 4.

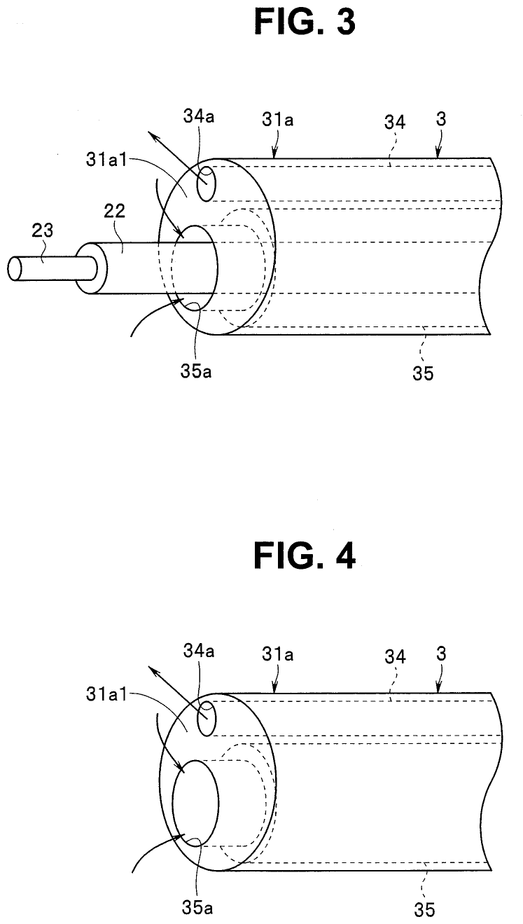

FIG. 3 is a perspective view showing a state of the distal end portion 31a of the insertion section 31 of the endoscope 3 through which a laser probe 22 is inserted in the first embodiment. FIG. 4 is a diagram showing a state of the distal end portion 31a of the insertion section 31 of the endoscope 3 after the laser probe 22 is pulled out in the first embodiment. Note that, although the laser probe is inserted through a suction channel 35 in FIG. 3, the laser probe may be inserted through another channel or a channel for the laser probe may be separately provided and the laser probe may be inserted through the channel for the laser probe.

As shown in FIG. 3 and FIG. 4, a liquid feed channel 34 for conveying liquid and the suction channel 35 also functioning as a treatment instrument channel are inserted through the insertion section 31. The liquid feed channel 34 includes a channel opening 34a on a distal end face 31a1 of the distal end portion 31a. The suction channel 35 includes a channel opening 35a (a treatment instrument opening) on the distal end face 31a1 of the distal end portion 31a.

The bending section 31b is provided on the proximal end side of the distal end portion 31a and configured to be bendable in, for example, two directions or upward, downward, left, and right four directions. When the bending section 31b is bent, a direction of the distal end portion 31a changes and a direction of observation by the observation system and an irradiation direction of illumination light by the illumination system change. The bending section 31b is also bent in order to improve insertability of the insertion section 31 in the subject.

The tubular section 31c is a tubular part that couples a proximal end of the bending section 31b and a distal end of the operation section 32. The tubular section 31c may be a rigid form in which the insertion section 31 does not bend or may be a flexible form in which the insertion section 31 bends according to a shape of a subject into which the insertion section 31 is inserted. An endoscope including an insertion section in the rigid form is generally called rigid endoscope and an endoscope including an insertion section in the flexible form is generally called flexible endoscope. For example, a rigid endoscope and a flexible endoscope in a medical field are defined in ISO8600-1: 2015.

The operation section 32 is provided on the proximal end side of the insertion section 31 and is a part for performing various kinds of operation concerning the endoscope 3. The operation section 32 includes, for example, a grasping section 32a, a bending operation lever 32b, an operation button 32c, a channel opening 34b on the proximal end side of the liquid feed channel 34, a channel opening 35b on the proximal end side of the suction channel 35, and a suction tube connector 35c of the suction channel 35.

The grasping section 32a is a part for a surgeon to grasp the endoscope 3 with a hand.

The bending operation lever 32b is an operation device for performing operation for bending the bending section 31b using, for example, a thumb of the hand grasping the grasping section 32a.

The operation button 32c includes, for example, a liquid feed button and a suction button. The liquid feed button is an operation button for performing liquid feed to the distal end portion 31a side through the liquid feed channel 34. The suction button is an operation button for performing suction from the distal end portion 31a side through the suction channel 35. A plurality of operation buttons 32c may include, for example, a button switch for performing operation (release operation and the like) relating to image pickup.

The channel opening 34b on the proximal end side of the liquid feed channel 34 is provided on one side surface on a distal end side of the grasping section 32a. A liquid feed tube 53 is connected to the channel opening 34b.

The channel opening 35b on the proximal end side of the suction channel 35 is provided on the other side surface on the distal end side of the grasping section 32a. The laser probe 22 is inserted into the channel opening 35b of the suction channel 35 using a protective tube 24. The protective tube 24 prevents a bend of an optical fiber 23 included in the laser probe 22. Note that the suction channel 35 also functions as the treatment instrument channel as explained above and is used to allow insertion of various treatment instruments. Therefore, a treatment instrument such as forceps may be inserted through the suction channel 35 instead of the laser probe 22.

The suction channel 35 suctions liquid in the subject from the channel opening 35a together with a fractured calculus. A first suction tube 54 is connected to the suction tube connector 35c provided near the channel opening 35b on the proximal end side of the suction channel 35.

The universal cable 33 is extended from, for example, a side surface on the proximal end side of the operation section 32 and connected to the endoscope control apparatus 4.

The endoscope control apparatus 4 also functions as an image processing apparatus and a light source apparatus, controls the endoscope 3, processes an image pickup signal acquired from the endoscope 3, and supplies illumination light to the endoscope 3. The endoscope control apparatus 4 performs image processing on the image pickup signal and generates an image signal that can be displayed. The image signal generated by the endoscope control apparatus 4 is outputted to a monitor 4a and an endoscopic image is displayed on the monitor 4a. Note that the image processing apparatus and the light source apparatus may be respectively configured by different bodies.

The laser system 2 is a calculus fracturing apparatus including a laser light source apparatus 21 and the laser probe 22. The laser light source apparatus 21 generates laser light (energy) for fracturing a calculus (a urinary calculus) present in a urinary tract such as a kidney, a ureter, a bladder, and a urethra and supplies the laser light to the laser probe 22. The laser probe 22 includes the optical fiber 23 that transmits the laser light. A distal end of the laser probe 22 is extended from the channel opening 35a on the distal end side of the suction channel 35 and the laser light is generated by the laser light source apparatus 21. Then, the laser light transmitted by the optical fiber 23 of the laser probe 22 is irradiated on a calculus in the subject from a distal end of the optical fiber 23 and the calculus is fractured.

Note that, in the present embodiment, the laser system 2 is an example of the calculus fracturing apparatus. However, the calculus fracturing apparatus is not limited to this and only has to be an apparatus that can fracture a calculus. For example, when the calculus is fractured using ultrasound, an ultrasonic probe and an ultrasonic apparatus that supplies energy to the ultrasonic probe may be used as the calculus fracturing apparatus instead of the laser probe 22 and the laser light source apparatus 21.

The perfusion system 1 includes a perfusion system main body 10 including a first pump 13 that is a liquid feed pump, a second pump 14 that is a suction pump, and a pressure gauge 19, a liquid feed source 51, a waste liquid tank 52, the liquid feed tube 53, the first suction tube 54, a first filter 55, a second filter 56, and a second suction tube 57.

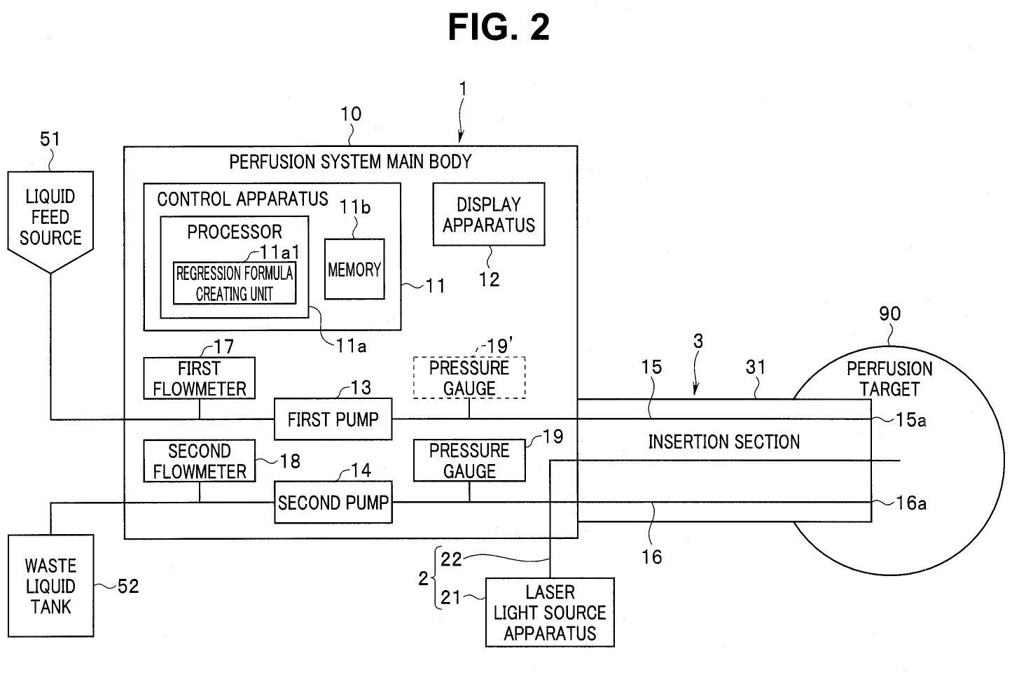

FIG. 2 is a diagram showing a configuration example of the perfusion system 1 in the first embodiment.

As shown in FIG. 2, the perfusion system main body 10 further includes a control apparatus 11, a display apparatus 12, a first flowmeter 17, and a second flowmeter 18. The perfusion system 1 further includes a liquid feed passage 15 including a liquid feed port 15a at a distal end and a suction passage 16 including a suction port 16a at a distal end. Note that the perfusion system main body 10 may include a pressure gauge 19′ as explained below. Order of disposing the flowmeters, the pressure gauges, and the pumps is not limited to order shown in the figure.

The liquid feed source 51 stores liquid to be fed into the subject. The liquid stored in the liquid feed source 51 is, for example, saline.

The liquid feed source 51 is connected to the first pump 13 by the liquid feed tube 53. The liquid feed tube 53 on the distal end side relative to the first pump 13 is connected to the channel opening 34b on the proximal end side of the liquid feed channel 34.

The first pump 13 is a liquid feed pump that feeds, to the liquid feed passage 15, the liquid supplied from the liquid feed source 51. When the first pump 13 operates, the liquid in the liquid feed source 51 is fed passing through the liquid feed tube 53 and discharged from the channel opening 34a on the distal end side into the perfusion target 90 of the subject passing through the liquid feed channel 34.

Therefore, the liquid feed passage 15 includes the liquid feed tube 53 and the liquid feed channel 34. The channel opening 34a functions as the liquid feed port 15a at a distal end of the liquid feed passage 15. However, the liquid feed tube 53 may be inserted into the liquid feed channel 34 and projected from the distal end face 31a1 of the distal end portion 31a. A distal end of the liquid feed tube 53 may be used as the liquid feed port 15a.

When being connected to the suction tube connector 35c, the first suction tube 54 communicates with an inside of the suction channel 35. The first suction tube 54 is connected to the pressure gauge 19 and the second filter 56 through the first filter 55.

The first filter 55 and the second filter 56 are appliances that filter a calculus and a mucous membrane suctioned from the perfusion target 90. Of the first filter 55 and the second filter 56, for example, the first filter 55 is used to collect a calculus. The first filter 55 is attached to, for example, the distal end side of the grasping section 32a in the operation section 32 of the endoscope 3. However, the first filter 55 and the second filter 56 are not limited to disposition shown in FIG. 1. One of the first filter 55 and the second filter 56 may be provided or both of the first filter 55 and the second filter 56 may not be provided.

The pressure gauge 19 is provided on the suction passage 16 and includes a pressure sensor. The pressure gauge 19 detects pressure in the suction passage 16. In the configuration example shown in FIG. 1, the pressure gauge 19 detects pressure applied to liquid suctioned by the first suction tube 54.

One end of the second suction tube 57 is connected to the second filter 56. The other end of the second suction tube 57 is connected to the waste liquid tank 52 through the second pump 14. Therefore, the suction passage 16 includes the suction channel 35, the first suction tube 54, and the second suction tube 57. The channel opening 35a functions as the suction port 16a at a distal end of the suction passage 16. However, the first suction tube 54 may be inserted into the suction channel 35 and projected from the distal end face 31a1 of the distal end portion 31a. A distal end of the first suction tube 54 may be used as the suction port 16a.

The second pump 14 is a suction pump that suctions the liquid in the perfusion target 90 together with a calculus and feeds the liquid to the waste liquid tank 52 side through the suction passage 16. The first pump 13 and the second pump 14 are driven by the control apparatus 11 independently of each other. Note that the second pump 14 may be disposed at a rear end of the waste liquid tank 52.

Since the first pump 13 and the second pump 14 are driven independently of each other, as operations of the perfusion system 1, there are a first operation (a first operation mode), a second operation (a second operation mode), a third operation (a third operation mode), and off (an OFF mode).

In the first operation mode, the first pump 13 operates and feeds liquid and the second pump 14 does not operate and does not suction the liquid (only the liquid feed is performed). In the second operation mode, the first pump 13 operates and feeds the liquid and the second pump 14 operates and suctions the liquid (the liquid feed and the suction are performed). In the third operation mode, the first pump 13 does not operate and does not feed the liquid and the second pump 14 operates and suctions the liquid (only the suction is performed). In the OFF mode, both of the first pump 13 and the second pump 14 are off and do not operate (the liquid feed and the suction are not performed).

When the second pump 14 operates, the second pump 14 suctions the liquid in the perfusion target 90 together with a calculus from the channel opening 35a through the channel opening 35a of the suction channel 35.

Even when the laser probe 22 is inserted through the suction channel 35, since a gap is present between the laser probe 22 and the suction channel 35, suction by the second pump 14 is possible. Therefore, collection of a fractured calculus by perfusion may be performed in any of a case in which the laser probe 22 is inserted through the suction channel 35 (FIG. 3) and a case in which the laser probe 22 is pulled out from the suction channel 35 (FIG. 4).

In a state shown in FIG. 3, the calculus and the liquid are suctioned from a gap between the channel opening 35a and the laser probe 22. In a state shown in FIG. 4, since the laser probe 22 is pulled out, a sectional area of a portion where the liquid passes in the suction channel 35 increases. Therefore, the calculus can be collected together with the liquid in a shorter time in the state shown in FIG. 4 compared with the state shown in FIG. 3.

In the second operation, when the first pump 13 and the second pump 14 are caused to simultaneously operate, the liquid feed into the perfusion target 90 through the liquid feed channel 34 and the suction of the liquid in the perfusion target 90 through the suction channel 35 are simultaneously performed. The liquid discharged from the liquid feed channel 34 generates a flow (perfusion) circulating in the perfusion target 90 to allow a fractured calculus to be carried on the flow, whereby calculus collection efficiency by the perfusion system 1 is improved.

The first flowmeter 17 is provided on the liquid feed passage 15 and detects a liquid feed flow rate in the liquid feed passage 15. A processor 11a of the control apparatus 11 acquires the liquid feed flow rate detected by the first flowmeter 17.

The second flowmeter 18 is provided on the suction passage 16 and detects a suction flow rate in the suction passage 16. The processor 11a acquires the suction flow rate detected by the second flowmeter 18.

The control apparatus 11 includes the processor 11a and a memory 11b.

The processor 11a includes, for example, hardware such as an ASIC (application specific integrated circuit) or an FPGA (field programmable gate array) including a CPU (central processing unit).

The memory 11b is a storage device (or a recording medium) that stores a processing program to be executed by the processor 11a, various setting values, and the like.

Note that, here, an example is explained in which the processor 11a reads and executes the processing program stored in the memory 11b to thereby perform functions of respective units. However, not only this, but, for example, at least a part of the functions of the respective units performed by the control apparatus 11 may be configured as a dedicated electronic circuit.

The processor 11a includes a regression formula creating unit 11a1 as a functional unit.

The display apparatus 12 displays various pieces of information concerning the perfusion system 1. Note that the display apparatus 12 may be used also for display of information concerning the laser system 2.

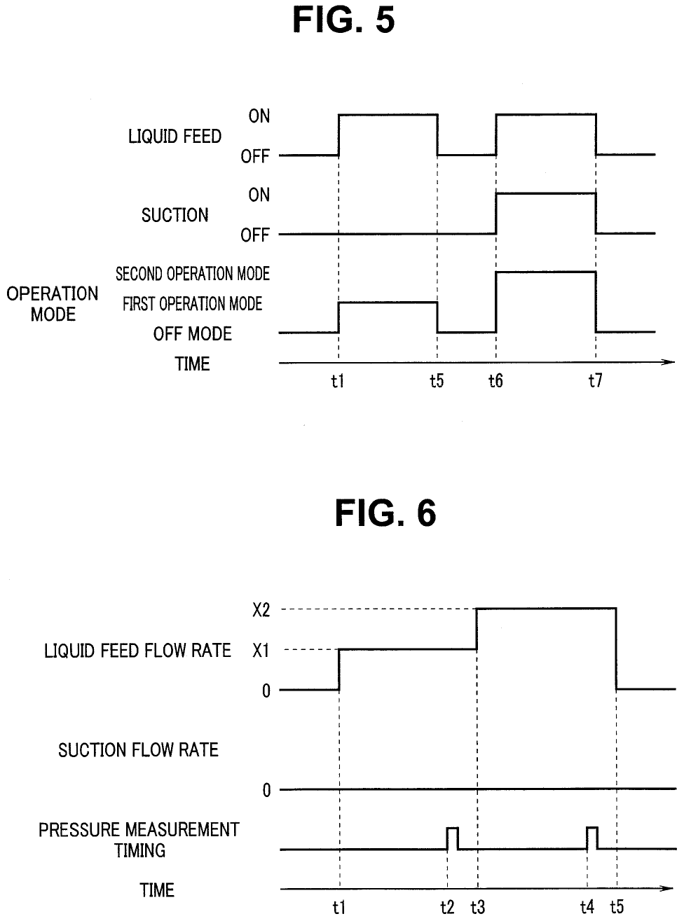

FIG. 5 is a timing chart showing a flow of processing for performing the first operation and calculating a regression formula prior to performing the second operation in the perfusion system 1 in the first embodiment. FIG. 6 is a timing chart showing a flow of processing of the perfusion system 1 at the time when the first operation is performed in order to calculate a regression formula in the first embodiment.

First, the surgeon inserts the insertion section 31 of the endoscope 3 into the subject and disposes the liquid feed port 15a at the distal end of the liquid feed passage 15 and the suction port 16a at the distal end of the suction passage 16 in the perfusion target 90. Consequently, a calculus can be collected from an organ of the perfusion target 90 by perfusion action.

As shown in FIG. 5, the processor 11a performs the first operation at least once prior to performing the second operation for collecting a calculus and calculates a regression formula. For example, when the perfusion target 90 is an organ (a kidney or the like) that is in a shrunk state in the beginning, the surgeon sometimes desires to swell the perfusion target 90 in order to observe an inside of the perfusion target 90 with an endoscopic image. In this case, since the processor 11a performs the first operation for feeding liquid without suctioning the liquid, the processor 11a may calculate the regression formula at this time. The processor 11a may, of course, calculate the regression formula separately from an operation for swelling the perfusion target 90.

In examples shown in FIG. 5 and FIG. 6, both of the first pump 13 and the second pump 14 are off (in the OFF mode) before a time point t1. At the time point t1 to a time point t5, the processor 11a turns on the first pump 13 while keeping the second pump 14 off and the perfusion system 1 operates in the first operation mode.

When calculating the regression formula in the first operation mode, the processor 11a controls the first pump 13 to feed the liquid at two or more liquid feed flow rates having different values.

As shown in FIG. 6, at the time point t1, the processor 11a sets a liquid feed flow rate of the first pump 13 to a first liquid feed flow rate X1 while keeping the second pump 14 off. The processor 11a performs liquid feed at the first liquid feed flow rate X1 (a constant liquid feed flow rate) and, after waiting for perfusion in the perfusion target 90 to stabilize, that is, pressure in the perfusion target 90 to stabilize, at the time point t2, detects pressure P1 in the suction passage 16 with the pressure gauge 19. Since the second pump 14 is off, there is no liquid flow in the suction passage 16 and the pressure gauge 19 detects a hydrostatic pressure. Therefore, the pressure P1 detected by the pressure gauge 19 (more accurately, the pressure P1 corrected according to a difference between heights in a gravity direction of the pressure gauge 19 and the suction port 16a) can be regarded as the pressure in the perfusion target 90. The processor 11a acquires the pressure P1 detected by the pressure gauge 19.

At the time point t3, the processor 11a sets the liquid feed flow rate of the first pump 13 to a second liquid feed flow rate X2 having a value different from the liquid feed flow rate X1 (here, for example, X2>X1). The processor 11a performs liquid feed at the second liquid feed flow rate X2 (a constant liquid feed flow rate) and, after waiting for the perfusion and the pressure in the perfusion target 90 to stabilize, at the time point t4, detects pressure P2 in the suction passage 16 with the pressure gauge 19. The pressure P2 detected by the pressure gauge 19 (like P1, more accurately, the corrected pressure P2) can be regarded as the pressure in the perfusion target 90 as explained above. The processor 11a acquires the pressure P2 detected by the pressure gauge 19.

In this way, the processor 11a acquires the liquid feed flow rate in the liquid feed passage 15 and the pressure in the suction passage 16 at the time of the first operation for feeding the liquid to the perfusion target 90 with the liquid feed passage 15 and not suctioning the liquid from the perfusion target 90 with the suction passage 16.

The regression formula creating unit 11a1 calculates a regression formula for giving a relation between the liquid feed flow rate and the pressure in the suction passage 16 at the time of the first operation.

More specifically, when x represents any liquid feed flow rate and y represents pressure in the suction passage 16 corresponding to x, the regression formula creating unit 11a1 of the processor 11a calculates a regression line indicated by Equation (1) as a regression formula based on the pressure P1 (mmHg) detected by the pressure gauge 19 at the first liquid feed flow rate X1 (mL/min) and the pressure P2 (mmHg) detected by the pressure gauge 19 at the second liquid feed flow rate X2 (mL/min).

y=ax+b (1)

Here, coefficients “a” and “b” are respectively given by the following Equations (2) and (3).

a=(P2−P1)/(X2−X1) (2)

b=P1/(aX1) (3)

The processor 11a stores the acquired regression line in, for example, the memory 11b.

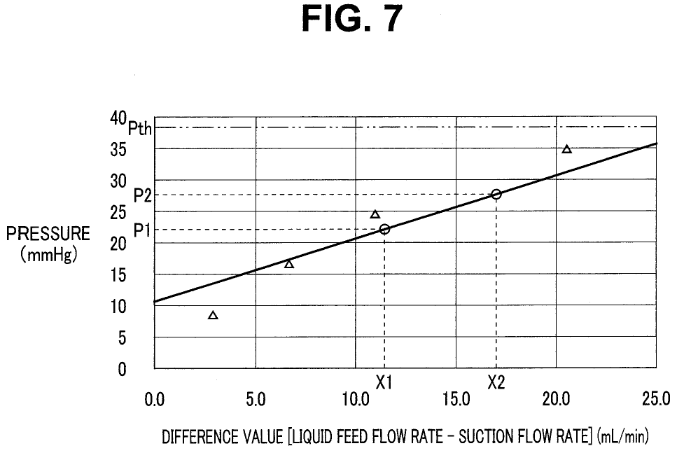

FIG. 7 is a graph showing an example of a regression formula calculated by the processor 11a in the first embodiment. The regression line y=ax+b calculated as explained above is a straight line passing (X1, P1) and (X2, P2) as shown in FIG. 7.

When specific values are given to the coefficients “a” and “b” of the regression line of the example shown in FIG. 7, the regression line changes to the following Equation (4).

y=x+10.7 (4)

Triangle marks in FIG. 7 indicate a result obtained when a pressure sensor or the like is disposed in the perfusion target 90 and pressure inside the perfusion target 90 (internal pressure) is directly detected in a different method in order to measure accuracy of the regression line y=ax+b. As shown in the figure, the regression line y=ax+b gives appropriate approximate values for measurement values.

Note that, in the above explanation, the regression line is calculated based on a set of two sets of the liquid feed flow rates X1 and X2 having the different values and the pressures P1 and P2 in the suction passage 16. However, more generally, n sets (n≥2) of a liquid feed flow rate X (X1, X2, Xn) and pressure P (P1, P2, . . . , Pn) may be acquired in the first operation mode and an optimized regression line may be calculated using, for example, a method of least squares based on data of the n sets.

Further, the regression formula is not limited to the regression line. More generally, a regression curve for giving pressure y in the suction passage 16 for any liquid feed flow rate X may be calculated as the regression formula. Since the regression line is primary approximation of the regression curve, a secondary or higher degree regression curve may be calculated and consistency with measurement values may be improved.

When the regression formula is acquired, the first operation mode is ended at the time point t5 and, in the example shown in FIG. 5, turns off the first pump 13 and the second pump 14 once (however, the processor 11a may directly shift from the first operation mode to the second operation mode not through the OFF mode or, when it is desired to further swell the perfusion target 90, may continue the first operation mode).

Thereafter, at a time point t6, the processor 11a turns on the first pump 13 and the second pump 14 and enters the second operation mode, feeds the liquid to the perfusion target 90 with the liquid feed passage 15 at a liquid feed flow rate set by the surgeon, and suctions the liquid from the perfusion target 90 with the suction passage 16 at a suction flow rate set by the surgeon. Consequently, perfusion is generated in the perfusion target 90 and a calculus is suctioned into the suction passage 16 from the suction port 16a together with the liquid. The calculus suctioned into the suction passage 16 is collected by, for example, the first filter 55 as explained above.

At the time of the second operation mode, the first flowmeter 17 detects a liquid feed flow rate X (X≥0) in the liquid feed passage 15 and the second flowmeter 18 detects a suction flow rate Z (Z≥0) in the suction passage 16. At the time of the second operation, the processor 11a acquires the liquid feed flow rate X from the first flowmeter 17, acquires the suction flow rate Z from the second flowmeter 18, and subtracts the suction flow rate Z from the liquid feed flow rate X to acquire a flow rate difference (X−Z).

The processor 11a substitutes the flow rate difference (X−Z) in x (x=(X−Z)) of the regression formula (here, the regression line) explained above stored in the memory 11b to calculate pressure y and sets the calculated pressure y as an estimation value of an internal pressure of the perfusion target 90 at the time of the second operation when the liquid feed flow rate is X and the suction flow rate is Z. In this way, the estimation value calculated from the flow rate difference (X−Z) based on the regression formula is displayed on, for example, the display apparatus 12. The surgeon can recognize the estimation value as the internal pressure of the perfusion target 90.

The surgeon changes at least one of a liquid feed flow rate or a suction flow rate of the perfusion system 1 as appropriate such that a swelling of the perfusion target 90 is secured in order to secure a surgical field and such that a calculus is appropriately carried by liquid. Further, the surgeon changes disposition, a direction, and the like of the liquid feed port 15a of the liquid feed passage 15 and the suction port of the suction passage 16 according to necessity while observing a state of calculus collection with the monitor 4a of the endoscope control apparatus 4.

The processor 11a acquires a flow rate difference every time at least one of the liquid feed flow rate X or the suction flow rate Z changes, calculates an estimation value of the internal pressure of the perfusion target 90 based on the regression line stored in the memory 11b, and displays the estimation value on the display apparatus 12.

Consequently, the surgeon can always grasp an estimation value of the internal pressure in a combination of the liquid feed flow rate X having any value and the suction flow rate Z having any value.

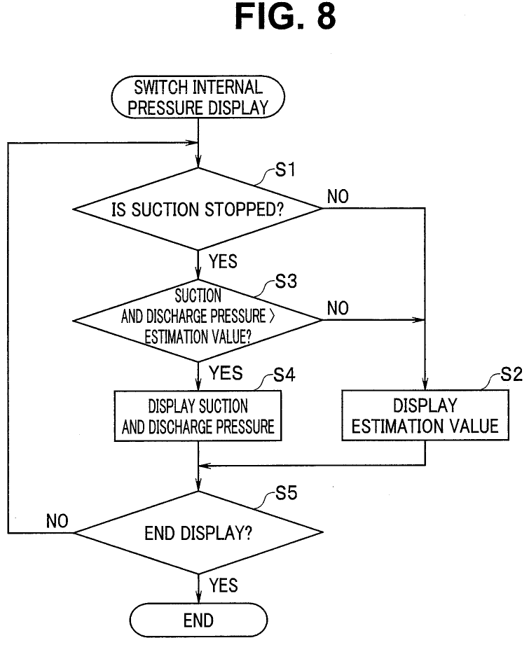

FIG. 8 is a flowchart showing processing for switching display of an internal pressure in the perfusion system 1 in the first embodiment.

When the processing is started, the processor 11a determines whether suction by the second pump 14 is stopped (step S1). Here, when determining that the suction is performed, the processor 11a displays, on the display apparatus 12, an estimation value based on the regression formula as an internal pressure of the perfusion target 90 (step S2). When the estimation value based on the regression formula is displayed, a graph indicating the regression formula may be displayed on the display apparatus 12. In this way, the internal pressure of the perfusion target 90 is displayed as the estimation value during a procedure in which liquid feed and suction are concurrently used.

In contrast, there is a case in which the suction is stopped during the procedure. For example, a case in which the suction is not performed and the liquid feed is performed, for example, it is desired to swell the perfusion target 90 or move a calculus corresponds to the case in which the suction is stopped. A case in which the suction and the liquid feed are not performed in order to observe a state of the inside of the perfusion target 90 with an endoscopic image also corresponds to the case in which the suction is stopped.

In such a case, when the processor 11a determines in step S1 that the suction is stopped, the processor 11a acquires pressure (suction and discharge pressure) in the suction passage 16 detected by the pressure gauge 19. The processor 11a compares the estimation value based on the regression formula and the suction and discharge pressure (step S3). Note that, here, since the suction is stopped (the suction flow rate Z=0), the flow rate difference is X. The pressure y calculated based on the regression formula by substituting X in x (x=X) is the estimation value.

Here, when determining that the suction and discharge pressure is equal to or smaller than the estimation value, processing in step S2 is performed. The estimation value is displayed on the display apparatus 12 as the internal pressure by control of the processor 11a. This is because the suction and discharge pressure of the measurement value detected by the pressure gauge 19 immediately after the second pump 14 is stopped is sometimes a negative pressure based on an air pressure because influence of a suction pressure by the second pump 14 still remains.

On the other hand, when determining in step S3 that the suction and discharge pressure is larger than the estimation value, the processor 11a performs control to display the suction and discharge pressure as the internal pressure on the display apparatus 12 (step S4). The processor 11a performs processing in step S2 or step S4 according to a determination result of step S3 in this way to display not-smaller one of the estimation value and the suction and discharge pressure on the display apparatus 12 (that is, the processor 11a may display a larger value when the values are different and may display any of the values when the values are the same). Consequently, a value smaller than an actual internal pressure of the perfusion target 90 is prevented from being displayed and safety is improved.

Note that, in step S3, instead of comparing the estimation value and the suction and discharge pressure, the processor 11a may determine whether a value of the suction and discharge pressure detected by the pressure gauge 19 is positive based on the air pressure. The processor 11a may perform control to display the estimation value in step S2 when determining that the value of the suction and discharge pressure is negative and display the suction and discharge pressure in step S4 when determining that the value of the suction and discharge pressure is positive. When displaying the estimation value in step S2 and when displaying the suction and discharge pressure in step S4, the processor 11a may clearly indicate to which of the values a display value belongs.

The surgeon can adjust the internal pressure to get close to a target value (for example, a value set by the surgeon as an optimum internal pressure) (not to deviate from a predetermined range in which the internal pressure is determined as normal) by viewing the internal pressure and the graph displayed on the display apparatus 12 and changing at least one of the liquid feed flow rate or the suction flow rate.

After performing the processing in step S2 or step S4, the processor 11a determines whether to end the display (step S5). When determining not to end the display, the processor 11a returns to step S1 and performs the processing explained above.

Pth in FIG. 7 indicates a predetermined threshold for the internal pressure of the perfusion target 90. The predetermined threshold Pth is, for example, a value set by the surgeon to determine that the internal pressure of the perfusion target 90 is an abnormal value when the internal pressure exceeds the predetermined threshold Pth. A specific example of the predetermined threshold Pth is a value exceeding a range of an internal pressure that can change in a normal perfusion state (for example, a value 20% higher than the target value).

When the estimation value is larger than the predetermined threshold Pth or when a state in which the estimation value is larger than the predetermined threshold Pth continues for a predetermined time or longer, the processor 11a may display, for example, on the display apparatus 12, a notification (warning) indicating that the internal pressure of the perfusion target 90 is abnormal and perform flashing display according to necessity or may further emit buzzer sound or the like. According to presentation of such information, the surgeon can recognize occurrence of the abnormality and adjust at least one of the liquid feed flow rate or the suction flow rate to be the predetermined threshold Pth or less.

Note that the adjustment for bring the internal pressure (the estimation value) close to the target value is not limited to be manually performed by the surgeon. The processor 11a may perform feedback control of at least one of the first pump 13 or the second pump 14 to automatically perform the adjustment. Alternatively, when the surgeon changes the setting of the liquid feed flow rate or the suction flow rate, the processor 11a may perform control, based on the regression formula, to maintain a predetermined flow rate difference such that the internal pressure (the estimation value) reaches the target value.

In this case, when the internal pressure (the estimation value) is larger than the target value, the processor 11a adjusts at least one of the liquid feed flow rate or the suction flow rate (including a case in which at least one of the first pump 13 and the second pump 14 is set zero) such that the flow rate difference decreases. When the internal pressure (the estimation value) is smaller than the target value, the processor 11a adjusts at least one of the liquid feed flow rate or the suction flow rate such that the flow rate difference increases. Note that, when the automatic adjustment is performed, the estimation value is displayed as a numerical value or a graph on a real time basis together with the predetermined threshold Pth.

Thereafter, the surgeon determines that the collection of the calculus by the perfusion has ended, for example, at a time point t7, the processor 11a ends the second operation mode. Note that, when the liquid still remains in the perfusion target 90 at this time, the processor 11a performs only the suction in the third operation mode and suctions and collects the liquid. In this way, when determining in step S5 to end the display, the processor 11a ends the processing shown in FIG. 8.

Note that the internal pressure, the graph indicating the regression formula, the warning display, and the like are not limited to be displayed on the display apparatus 12 and may be displayed on the monitor 4a of the endoscope control apparatus 4, may be displayed on a monitor 12 of the laser system 2, or may be displayed on another external monitor or the like. Alternatively, only a signal relating to the warning may be transmitted to another system at a warning time. For example, the signal relating to the warning may be transmitted to the laser system 2 at the warning time and a laser output may be limited or stopped by control on the laser system 2 side.

Although one predetermined threshold Pth is shown in FIG. 7, a plurality of thresholds having different values may be set. For example, a pressure value of [(the air pressure)+30 (mmHg)] may be set as a first threshold, a pressure value of [(the air pressure)+100 (mmHg)] may be set as a second threshold, and a display color and warning sound (tone, volume, and the like) may be differentiated according to a magnitude relation between the internal pressure and the respective thresholds.

More specifically, for example, when the internal pressure is the first threshold of [(the air pressure)+30 (mmHg)] or smaller, a numerical value may be displayed in green, when the internal pressure is larger than the first threshold and equal to or smaller than the second threshold of [(the air pressure)+100 (mmHg)], the numerical value may be displayed in yellow, and, when the internal pressure is larger than the second threshold, the numerical value is displayed in red. Further, for example, when the internal pressure is [(the air pressure)+α (mmHg)] (where α<0 and a negative pressure lower than the air pressure) at which the perfusion target 90 such as a kidney is in a shrunk state, for example, the numerical value may be displayed in, for example, blue.

Only the estimation value may be displayed without the color being differentiated. Only the color may be differentiated and displayed without the estimation value (the numerical value) being displayed.

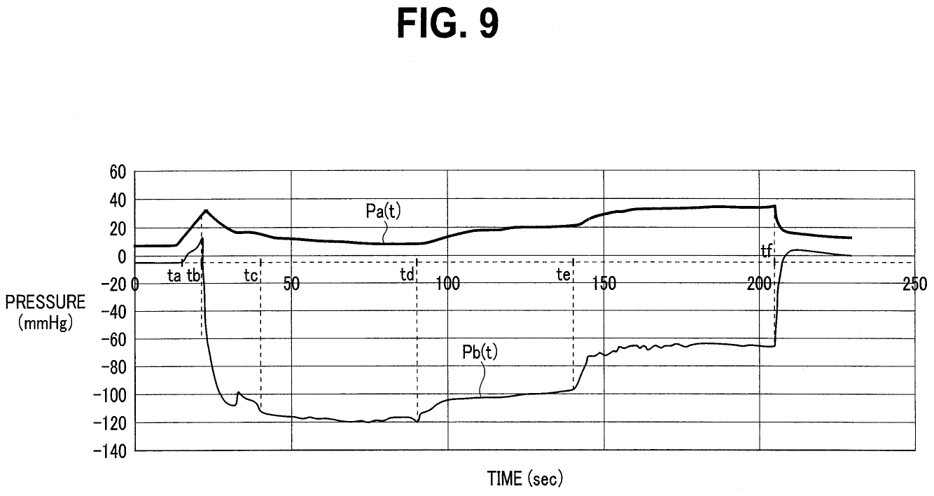

FIG. 9 is a time chart showing an example in which pressure detected by the pressure gauge 19 changes when a liquid feed flow rate and a suction flow rate do not change in the first embodiment. Note that, in FIG. 9, a pressure difference from an air pressure at the time when the air pressure is set as a reference (pressure=0 (mmHg)) is shown.

A graph Pa(t) in FIG. 9 indicates a temporal change in an internal pressure of the perfusion target 90 directly detected by disposing a pressure sensor or the like in the perfusion target 90 in a different method. A graph Pb(t) indicates a temporal change in pressure in the suction passage 16 detected by the pressure gauge 19.

In the example shown in FIG. 9, the perfusion system 1 is in the OFF mode before a time point ta and does not perform both of liquid feed and suction. At this time, the graph Pa(t) indicates a negative pressure value of, for example, approximately −4 (mmHg) and the graph Pb(t) indicates a pressure value of, for example, approximately 8 (mmHg).

Thereafter, at the time point ta, the perfusion system 1 starts liquid feed at a constant liquid feed flow rate. According to the liquid feed, values of both of the graph Pa(t) and the graph Pb(t) increase while substantially maintaining a difference between the values and the graph Pb(t) also indicates a positive pressure value.

At a time point tb slightly later than the time point ta, the perfusion system 1 starts suction at a constant suction flow rate. By somewhat delaying the suction start relative to the liquid feed start, an amount of the liquid in the perfusion target 90 increases and the perfusion target 90 swells. When the suction is started, the values of both of the graph Pa(t) and the graph Pb(t) decrease. In the example shown in FIG. 9, since the first pump 13 and the second pump 14 are operating in a state of the liquid feed flow rate>the suction flow rate, the graph Pa(t) indicating an actual internal pressure of the perfusion target 90 maintains a positive pressure value. On the other hand, the graph Pb(t) affected by a suction pressure by the second pump 14 shifts from a positive pressure value to a negative pressure value immediately after starting suction at the time point tb.

When the liquid feed and the suction is performed in the state of the liquid feed flow rate>the suction flow rate, the liquid is stored inside the perfusion target 90 and the perfusion target 90 such as the kidney further swells. When the perfusion target 90 swells to a constant size, a shrinking pressure of the perfusion target 90 and pressure pressing an internal wall of the perfusion target 90 are balanced and the perfusion target 90 cannot swell more. The liquid having a rate of (the liquid feed flow rate—the suction flow rate) leaks to a region other than a region where perfusion is implemented (for example, the proximal end side of a passage in the perfusion system 1) as a “leak amount”.

When the liquid feed and the suction are performed for a predetermined time and a time point tc is reached, the liquid feed, the suction, and the leak reach steady states. The graph Pb(t) (and the graph Pa(t)) indicates a substantially constant value.

Thereafter, although the liquid is fed at the constant liquid feed flow rate and is suctioned at the constant suction flow rate, the value of the graph Pb(t) (and the graph Pa(t)) changes at a time point td and the value of the graph Pb(t) (and the graph Pa(t)) further changes at a time point te.

Further, thereafter, when the liquid feed and the suction are stopped at a time point tf, the value of the graph Pb(t) rapidly increases and, after indicating, for example, a positive pressure value once, gradually comes close to the pressure value at the time of the OFF mode before the time point ta. The value of the graph Pb(t) also gradually gets close to the pressure value at the time of the OFF mode before the time point ta after rapidly decreasing.

In the operation example shown in FIG. 9, although the first pump 13 and the second pump 14 are operating at the constant liquid feed flow rate and the constant suction flow rate from the time point tb to the time point tf, the pressure in the suction passage 16 detected by the pressure gauge 19 (the graph Pb(t)) suddenly changes at the time point td and the time point te after reaching the steady state at the time point tc. Such a sudden change in the pressure occurs, for example, when a constant leak amount at the time points tc to td (or the time points td to te) decreases at the time point td (or the time point te). A change in the leak amount occurs, for example, when a posture of a patient during an operation changes or an access sheath used in inserting the insertion section 31 is moved.

When the steady leak amount changes, in some case, a correct estimation value cannot be obtained by the regression formula acquired before the perfusion is started and an estimation error occurs. When a fractured calculus is collected in a specific position in the perfusion target 90, in some case, the suction is not performed and only the liquid feed is performed by the first operation. In such a case as well, an estimation error of the regression formula occurs.

When the liquid feed flow rate and the suction flow rate are constant and do not change, the processor 11a monitors pressure in the suction passage 16 detected by the pressure gauge 19 and, when the pressure changes by a predetermined pressure difference or more, determines that accuracy of the regression formula is deteriorated.

The processor 11a displays a notification for urging recreation of the regression formula, for example, on the display apparatus 12. The surgeon may manually perform the first operation for recreating the regression formula. However, the processor 11a may obtain an approval by the surgeon and perform the first operation in automatic processing. The processor 11a calculates the regression formula again based on two or more sets of liquid feed flow rates and pressures in the suction passage 16 acquired in a new first operation and stores the regression formula in the memory 11b.

Note that, when the estimation error of the regression formula is quantitative, the processor 11a may deal with the estimation error by correcting the regression formula by a change amount in pressure detected by the pressure gauge 19 under the constant liquid feed flow rate and the constant suction flow rate. In this case, it is unnecessary to perform the first operation for recreating the regression formula again. The processor 11a stores the corrected regression formula in the memory 11b.

When the accuracy of the regression formula is deteriorated under the constant liquid feed flow rate and the constant suction flow rate, the difference between the estimation value based on the regression formula and the pressure in the suction passage 16 detected by the pressure gauge 19 changes. Therefore, the processor 11a may monitor the difference between the estimation value and the detected pressure and, when the difference changes by a predetermined value or more, determine that the accuracy of the regression formula is deteriorated.

By recreating the regression formula or correcting the regression formula when the estimation error occurs in this way, it is possible to prevent the accuracy of the regression formula from being deteriorated and stably acquire a correct estimation value.

Note that, in the above explanation, the processor 11a acquires the liquid feed flow rate detected by the first flowmeter 17 and the suction flow rate detected by the second flowmeter 18. However, the processor 11a is not limited to this. For example, the processor 11a may, instead of acquiring the liquid feed flow rate detected by the first flowmeter 17, estimate and acquire a liquid feed flow rate based on an operation amount of the first pump 13 that feeds the liquid to the liquid feed passage 15. The processor 11a may also, instead of acquiring the suction flow rate detected by the second flowmeter 18, estimate and acquire a suction flow rate based on an operation amount of the second pump 14 that suctions the liquid from the suction passage 16.

According to such a first embodiment, the internal pressure of the perfusion target 90 is estimated by calculating the regression formula based on the liquid feed flow rate and the pressure in the suction passage 16 acquired at the time of the first operation and applying, to the regression formula, the flow rate difference obtained by subtracting the suction flow rate from the liquid feed flow rate acquired at the time of the second operation. Therefore, it is possible to grasp the internal pressure of the perfusion target 90 without disposing a pressure gauge in the perfusion target 90.

At this time, by calculating a regression curve corresponding to any flow rate difference, even if at least one of the liquid feed flow rate or the suction flow rate is changed as desired, it is possible to stably acquire the internal pressure of the perfusion target 90. When the regression curve is changed to a regression line, since the regression line can be calculated from two sets of liquid feed flow rates and pressures in the suction passage 16, a burden on the surgeon is reduced.

For a narrow region where it is difficult to dispose a pressure gauge like a ureter, an internal pressure of the perfusion target 90 can be estimated from a liquid feed flow rate and a suction flow rate. Therefore, the surgeon can safely perform perfusion while grasping the internal pressure.

### Modification

In the above explanation, the pressure gauge 19 is provided on the suction passage 16. However, as shown in FIG. 2, the pressure gauge 19′ may be provided on the liquid feed passage 15 instead of (or in addition to) the pressure gauge 19.

In a modification, the processor 11a may estimate an internal pressure of the perfusion target 90 as explained below. When the liquid is fed to the liquid feed passage 15 by the first pump 13, the first flowmeter 17 detects a liquid feed flow rate in the liquid feed passage 15 and the pressure gauge 19′ detects pressure in the liquid feed passage 15.

The processor 11a acquires the pressure in the liquid feed passage 15 detected by the pressure gauge 19′ and the liquid feed flow rate in the liquid feed passage 15 detected by the first flowmeter 17.

Pressure in the liquid feed port 15a at the distal end of the liquid feed passage 15 drops more than pressure detected by the pressure gauge 19′ halfway in the liquid feed passage 15. However, the pressure drop depends on the liquid feed flow rate.

Therefore, the processor 11a subtracts the pressure drop corresponding to the liquid feed flow rate detected by the first flowmeter 17 from the pressure detected by the pressure gauge 19′ to acquire an estimation value of pressure at the distal end of the liquid feed passage 15.

When perfusion is implemented, the liquid feed port 15a at the distal end of the liquid feed passage 15 is disposed inside the perfusion target 90. Therefore, the estimation value of the pressure acquired by the processor 11a is an estimation value of internal pressure of the perfusion target 90.

Note that, when the first pump 13 is off, a liquid flow is absent in the liquid feed passage 15 and the pressure gauge 19′ detects a hydrostatic pressure. Therefore, the pressure detected by the pressure gauge 19′ (more accurately, pressure corrected according to a difference between heights in the gravity direction of the pressure gauge 19′ and the liquid feed port 15a) can be regarded as pressure in the perfusion target 90. Therefore, even when the liquid is not fed, the internal pressure of the perfusion target 90 can be acquired by the pressure gauge 19′.

The internal pressure of the perfusion target 90 may be estimated by such a modification. According to the modification, the internal pressure of the perfusion target 90 can be estimated even if the second flowmeter 18 and the pressure gauge 19 are absent. Therefore, it is possible to further simplify a configuration from the first embodiment and reduce manufacturing cost of the perfusion system 1.

In addition to the configuration in the first embodiment, as described in Japanese Patent Application Laid-Open Publication No. 2020-518342, the perfusion system may include a pressure sensor (a third pressure gauge) provided at the distal end portion 31a of the insertion section 31 of the endoscope 3. Consequently, it is possible to dispose the pressure sensor in the perfusion target 90 and directly measure a pressure value in the perfusion target 90 with the perfusion system. In that case, the perfusion system can monitor a difference between the estimation value of the internal pressure and the directly measured pressure value and, when the difference is larger than a predetermined threshold or when a state in which the difference is larger than the predetermined threshold continues for a predetermined time, determine that there is possibility of an abnormality of the pressure sensor provided at the distal end portion 31a or an abnormality in acquisition of the estimation value of the internal pressure (an abnormality of a sensor or the like of a pressure estimation system that acquires data for calculating the estimation value). The perfusion system can inform possibility of a failure to the surgeon based on a determination result.

Note that, in the above explanation, a case in which the present disclosure is the perfusion system is mainly explained. However, the present disclosure is not limited to this and may be an actuation method for the perfusion system, a control method for the perfusion system, a perfusion target internal pressure estimation method, or a living body internal pressure estimation method or may be a computer program for causing a computer to perform the same processing as these methods, a computer-readable non-transitory recording medium recording the computer program, and the like.

The present disclosure is not limited to the embodiment explained above per se. In an implementation stage, the constituent elements can be modified and embodied in a range not departing from the gist of the present disclosure. Various aspects of disclosures can be formed by appropriate combinations of a plurality of constituent elements disclosed in the embodiment. For example, several constituent elements may be deleted from all the constituent elements described in the embodiment. Further, the constituent elements described in different embodiments may be combined as appropriate. In this way, it goes without saying that various modifications and applications are possible within the range not departing from the gist of the disclosure.

What is claimed is:

1. A perfusion target internal pressure estimation method comprising:
at a time of a first operation for feeding liquid to a perfusion target with a liquid feed passage and not suctioning the liquid from the perfusion target with a suction passage, acquiring a liquid feed flow rate in the liquid feed passage and pressure in the suction passage;
at a time of a second operation for feeding the liquid to the perfusion target with the liquid feed passage and suctioning the liquid from the perfusion target with the suction passage, acquiring a liquid feed flow rate in the liquid feed passage and a suction flow rate in the suction passage;
subtracting the suction flow rate from the liquid feed flow rate to acquire a flow rate difference; and
acquiring, based on a regression formula of the liquid feed flow rate and the pressure in the suction passage at the time of the first operation, an estimation value of an internal pressure of the perfusion target at the time of the second operation from the flow rate difference.

2. The perfusion target internal pressure estimation method according to claim 1, further comprising:
at the time of the first operation, acquiring two or more pressures respectively in the suction passage at two or more liquid feed flow rates having different values;
calculating, based on acquired two or more sets of the liquid feed flow rates and the pressures in the suction passage, as the regression formula, a regression curve for giving pressure in the suction passage for any liquid feed flow rate; and
acquiring, based on the regression curve, as the estimation value, pressure in the suction passage corresponding to the liquid feed flow rate having a same value as the flow rate difference.

3. The perfusion target internal pressure estimation method according to claim 2, further comprising:
at the time of the first operation, when pressure in the suction passage acquired at a first liquid feed flow rate X1 is represented as P1, pressure in the suction passage acquired at a second liquid feed flow rate X2 is represented as P2, x represents any liquid feed flow rate, and y represents pressure in the suction passage corresponding to x, representing the regression curve as a regression line based on following relational expressions:
a=(P2−P1)/(X2−X1)
b=P1/(aX1)
y=ax+b; and
substituting the flow rate difference in x to acquire the estimation value.

4. The perfusion target internal pressure estimation method according to claim 2, further comprising: at the time of the second operation,
detecting and acquiring pressure in the suction passage with a pressure gauge provided on the suction passage; and
when the liquid feed flow rate and the suction flow rate do not change and the pressure in the suction passage changes by a predetermined pressure difference or more, performing the first operation and recalculating the regression formula.

5. The perfusion target internal pressure estimation method according to claim 1, further comprising bringing the estimation value close to a target value by:
when the estimation value is larger than the target value, adjusting at least one of the liquid feed flow rate or the suction flow rate such that the flow rate difference decreases; and
when the estimation value is smaller than the target value, adjusting at least one of the liquid feed flow rate or the suction flow rate such that the flow rate difference increases.

6. The perfusion target internal pressure estimation method according to claim 1, wherein
the liquid feed flow rate is acquired by a first flowmeter provided on the liquid feed passage or is estimated and acquired based on an operation amount of a first pump that feeds the liquid to the liquid feed passage, and
the suction flow rate is acquired by a second flowmeter provided on the suction passage or is estimated and acquired based on an operation amount of a second pump that suctions the liquid from the suction passage.

7. The perfusion target internal pressure estimation method according to claim 1, further comprising:
detecting and acquiring pressure in the suction passage with a pressure gauge provided on the suction passage; and
at the time of the first operation, displaying, on a display apparatus, a not-smaller value of the estimation value and the pressure in the suction passage acquired from the pressure gauge.

8. The perfusion target internal pressure estimation method according to claim 1, further comprising, when the estimation value is larger than a predetermined threshold, performing warning indicating that the internal pressure of the perfusion target is abnormal.

9. The perfusion target internal pressure estimation method according to claim 1, further comprising:
directly measuring a pressure value in the perfusion target with a pressure gauge disposed in the perfusion target; and
when a difference between the estimation value and the directly measured pressure value is larger than a predetermined threshold, performing warning indicating that there is an abnormality in the pressure gauge or the acquisition of the estimation value.

10. A perfusion system comprising:
a first pump configured to feed liquid to a liquid feed passage;
a first flowmeter configured to detect a liquid feed flow rate in the liquid feed passage;
a second pump configured to suction the liquid from a suction passage;
a second flowmeter configured to detect a suction flow rate in the suction passage;
a pressure gauge configured to detect pressure in the suction passage; and
a processor configured to control the first pump and the second pump based on information from the first flowmeter, the second flowmeter, and the pressure gauge, wherein
at a time of a first operation of controlling the first pump to feed the liquid to the liquid feed passage and a perfusion target and of controlling the second pump not to suction the liquid from the suction passage and the perfusion target, the processor is configured to perform:
acquiring the liquid feed flow rate detected by the first flowmeter and the pressure in the suction passage detected by the pressure gage, and
at a time of a second operation of controlling the first pump to feed the liquid to the liquid feed passage and the perfusion target and of controlling the second pump to suction the liquid from the suction passage and the perfusion target, the processor is configured to perform:
acquiring the liquid feed flow rate detected by the first flowmeter and the suction flow rate detected by the second flowmeter;
subtracting the suction flow rate from the liquid feed flow rate to acquire a flow rate difference; and
acquiring, based on a regression formula of the liquid feed flow rate and the pressure in the suction passage at the time of the first operation, an estimation value of an internal pressure of the perfusion target at the time of the second operation from the flow rate difference.

11. The perfusion system according to claim 10, wherein the processor:
at the time of the first operation, at two or more liquid feed flow rates having different values, acquires the two or more liquid feed flow rates and two or more pressures respectively in the suction passage;
calculates, based on acquired two or more sets of the liquid feed flow rates and the pressures in the suction passage, as the regression formula, a regression curve for giving pressure in the suction passage for any liquid feed flow rate; and
acquires, based on the regression curve, as the estimation value, pressure in the suction passage corresponding to the liquid feed flow rate having a same value as the flow rate difference.

12. The perfusion system according to claim 11, wherein the processor:
at the time of the first operation, when pressure in the suction passage acquired at a first liquid feed flow rate X1 is represented as P1, pressure in the suction passage acquired at a second liquid feed flow rate X2 is represented as P2, x represents any liquid feed flow rate, and y represents pressure in the suction passage corresponding to x, represents the regression curve as a regression line based on following relational expressions:
a=(P2−P1)/(X2−X1)
b=P1/(aX1)
y=ax+b; and
substitutes the flow rate difference in x to acquire the estimation value.

13. The perfusion system according to claim 11, wherein
the processor, at the time of the second operation:
acquires pressure in the suction passage detected by the pressure gauge; and
when the liquid feed flow rate and the suction flow rate do not change and the pressure in the suction passage changes by a predetermined pressure difference or more, performs the first operation and recalculates the regression formula.

14. The perfusion system according to claim 10, wherein the processor brings the estimation value close to a target value by:
when the estimation value is larger than the target value, adjusting at least one of the liquid feed flow rate or the suction flow rate such that the flow rate difference decreases; and
when the estimation value is smaller than the target value, adjusting at least one of the liquid feed flow rate or the suction flow rate such that the flow rate difference increases.

15. The perfusion system according to claim 10, wherein the processor:
instead of acquiring the liquid feed flow rate detected by the first flowmeter, estimates and acquires the liquid feed flow rate based on an operation amount of the first pump; and
instead of acquiring the suction flow rate detected by the second flowmeter, estimates and acquires the suction flow rate based on an operation amount of the second pump.

16. The perfusion system according to claim 10, wherein
the processor:
acquires pressure in the suction passage detected by the pressure gauge; and
at the time of the first operation, displays, on a display apparatus, a not-smaller value of the estimation value and the pressure in the suction passage acquired from the pressure gauge.

17. The perfusion system according to claim 10, wherein, when the estimation value is larger than a predetermined threshold, the processor performs warning indicating that the internal pressure of the perfusion target is abnormal.

18. A perfusion system comprising:
a liquid feed passage;
a pump configured to feed liquid to the liquid feed passage;
a pressure gauge provided on the liquid feed passage;
a first flowmeter provided on the liquid feed passage; and
a processor including hardware,
the processor: when the liquid is fed to the liquid feed passage by the pump, subtracting, from pressure in the liquid feed passage detected by the pressure gauge, a pressure drop corresponding to a liquid feed flow rate in the liquid feed passage detected by the first flowmeter and acquiring an estimation value of pressure at a distal end of the liquid feed passage.

19. A living body internal pressure estimation method comprising:
disposing a distal end of a liquid feed passage and a distal end of a suction passage in a living body;
performing a first operation for feeding liquid to the living body with the liquid feed passage and not suctioning the liquid from the living body with the suction passage;
performing a second operation for feeding the liquid to the living body with the liquid feed passage and suctioning the liquid from the living body with the suction passage; and
acquiring, based on a regression formula of a liquid feed flow rate of the liquid feed passage and pressure in the suction passage at a time of the first operation, an estimation value of an internal pressure of the living body at a time of the second operation from a flow rate difference obtained by subtracting a suction flow rate of the suction passage from a liquid feed flow rate of the liquid feed passage at the time of the second operation.
